(19) United States
(12) Patent Application Publication
(10) Pub. No.: US 2018/0055436 A1
(43) Pub. Date: Mar. 1, 2018

(54) FATIGUE DETECTION APPARATUS AND FATIGUE DETECTION METHOD

(71) Applicant: Winbond Electronics Corp., Taichung City (TW)

(72) Inventors: Chia-Chi Chang, Taichung City (TW); Hung-Yi Hsu, Taichung City (TW)

(73) Assignee: Winbond Electronics Corp., Taichung City (TW)

(21) Appl. No.: 15/399,721

(22) Filed: Jan. 5, 2017

(30) Foreign Application Priority Data
Aug. 30, 2016 (CN) 201610765712.1

(51) Int. Cl. A61B 5/18; A61B 5/0205; A61B 5/00

(52) U.S. Cl. CPC A61B 5/18; A61B 5/0205; A61B 5/0402; A61B 5/6823; A61B 5/7275

(57) ABSTRACT

A fatigue detection apparatus is provided. The fatigue detection apparatus includes a first detector, a second detector and a processor. The first detector is configured to obtain a first physiological signal and the second detector is configured to obtain a second physiological signal. The processor is coupled to the first detector and the second detector, obtains a plurality of characteristic time differences according to the first physiological signal and the second physiological signal, and determines a fatigue detection result according to a variation trend of the obtained characteristic time differences varying with time. Furthermore, a fatigue detection method is also provided.

### CROSS-REFERENCE TO RELATED APPLICATION

This application claims the priority benefit of China application serial no. 201610765712.1, filed on Aug. 30, 2016. The entirety of the above-mentioned patent application is hereby incorporated by reference herein and made a part of this specification.

### BACKGROUND OF THE INVENTION

### Field of the Invention

The invention relates to a detection apparatus and a detection method, particularly to a fatigue detection apparatus and a fatigue detection method.

### Description of Related Art

Traditionally, common techniques for detecting fatigue include two main categories: brain wave analysis and cardiovascular indicator analysis. A brain wave analysis mainly includes detecting whether a sleep wave has occurred in a brain wave so as to further determine a state of consciousness of a person under test. However, such method is subject to large individual differences and high complexity of brain waves, and has a characteristic of being easily interfered with by noise. As a result, the brain wave analysis has had low reliability and has not been well utilized so far. On the other hand, a cardiovascular indicator analysis mainly includes detecting a single physiological indicator relating to the heart and blood vessels so as to assess whether a person under test has shown signs of being asleep, such as slower heartbeat and so on, thereby determining whether the person under test has been in a fatigue state. In other words, by analyzing a heartbeat spectrum, it is known whether parasympathetic nerves of the person under test are being active, and whether the person under test is in the fatigue state is thereby determined. However, during analysis of the spectrum by such method, an object to be analyzed in the spectrum is likely to be distorted due to other variables (e.g., breathing) having a similar frequency. In addition, even if the person under test has entered the fatigue state, his/her heartbeat does not immediately slow down, and the insufficient real-time response capability also limits application of such method. Accordingly, persons skilled in the art are still striving for accurate and more real-time fatigue detection apparatus and method.

### SUMMARY OF THE INVENTION

The invention provides a fatigue detection apparatus and a fatigue detection method that are capable of accurately and in real time determining a variation in a user's consciousness.

The fatigue detection apparatus of the invention includes a first detector, a second detector and a processor. The first detector is configured to obtain a first physiological signal and the second detector is configured to obtain a second physiological signal. The processor is coupled to the first detector and the second detector.

The fatigue detection method of the invention includes the following steps. A first physiological signal is obtained. A second physiological signal is obtained. A plurality of characteristic time differences are obtained based on the first physiological signal and the second physiological signal. A fatigue detection result is determined based on a variation trend of the obtained characteristic time differences varying with time. This determination method is capable of determining a variation in a user's consciousness in an accurate and more real-time manner, and thus have wider application.

To make the above features and advantages of the invention more comprehensible, embodiments accompanied with drawings are explained in detail as follows.

### BRIEF DESCRIPTION OF THE DRAWINGS

FIG. 1 illustrates a schematic block view of a fatigue detection apparatus according to an embodiment of the invention.

FIG. 2 illustrates a schematic view of a fatigue detection apparatus for vehicles according to an embodiment of the invention.

FIG. 3 illustrates a flow chart of a fatigue detection method according to an embodiment of the invention.

FIG. 4 illustrates a schematic view of a fatigue detection method according to an embodiment of the invention.

### DETAILED DESCRIPTION OF DISCLOSED EMBODIMENTS

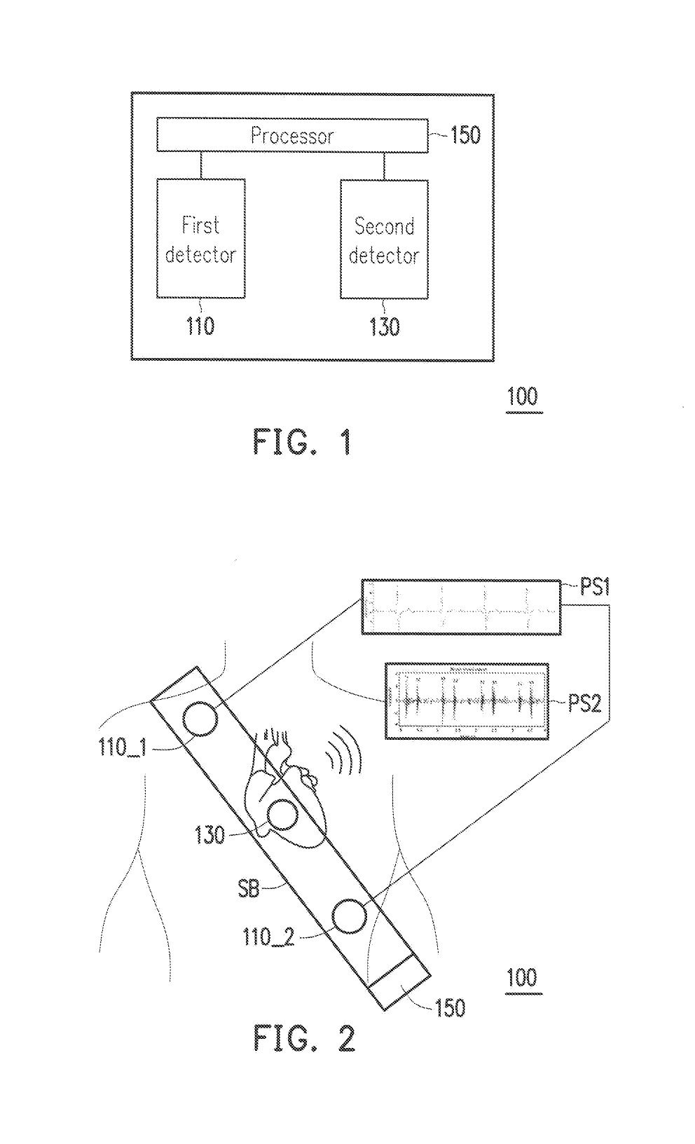

FIG. 1 illustrates a schematic block view of a fatigue detection apparatus according to an embodiment of the invention. Referring to FIG. 1, a fatigue detection apparatus 100 of the present embodiment includes a first detector 110, a second detector 130 and a processor 150. In the present embodiment, the first detector 110 is configured to obtain a first physiological signal corresponding to a first physiological indicator, and the second detector 130 is configured to obtain a second physiological signal corresponding to a second physiological indicator. The processor 150 is coupled to the first detector 110 and the second detector 130, performs a calculation based on the first physiological signal and the second physiological signal so as to obtain a plurality of characteristic time differences, and determines a fatigue detection result based on a variation trend of the obtained characteristic time differences varying with time.

The first detector 110 and the second detector 130 include, for example, a physiological signal detector configured to detect a physiological indicator such as blood pressure, body temperature, heart sound, cardiac electrical activity, pulse, breath or blood oxygen saturation so as to obtain a corresponding physiological signal. However, the invention is not limited thereto. In the present embodiment, the first detector 110 is, for example, an electrocardiogram (ECG) detector which detects a potential variation of different parts of a user's body surface by at least two electrodes, so as to obtain an ECG signal as the first physiological signal. The second detector 130 is, for example, a phonocardiogram (PCG) detector, which detects the user's heart sounds by a piezoelectric thin film, so as to obtain a PCG signal as the second physiological signal. However, the invention does not limit how the first detector 110 and the second detector 130 are actually implemented. In other words, persons of ordinary skill in the art may adjust, according to needs, how each of the aforesaid physiological signal detectors is actually implemented, so as to obtain a corresponding physiological signal.

It is worth mentioning that, in the present embodiment, the first detector 110 and the second detector 130 correspond respectively to different physiological indicators and obtain physiological signals of different types. In another embodiment, the first detector 110 and the second detector 130 may correspond to the same physiological indicator and obtain physiological signals of the same type. For example, PCG signals obtained from different positions on the user's body may correspond to shock waves emitted by different valves during operation of the user's heart. Therefore, the first detector 110 and the second detector 130 may also be, for example, PCG detectors installed in different positions on the user's body, so as to obtain the PCG signals emitted from different valves. Persons of ordinary skill in the art may determine the type of an obtained physiological signal according to their needs when implementing the invention. The invention does not impose any limitations on this.

The processor 150 is, for example, a central processing unit (CPU), a microprocessor, a digital signal processor (DSP), a field-programmable gate array (FPGA), a programmable logic device (PLD) or other similar device or a combination thereof. The invention is not limited thereto. In the present embodiment, the processor 150 is, for example, wiredly or wirelessly coupled to the first detector 110 and the second detector 130, and configured to obtain the first physiological signal and the second physiological signal, perform a calculation based on the first physiological signal and the second physiological signal so as to obtain a plurality of characteristic time differences, and determine a fatigue detection result based on a variation trend of the obtained characteristic time differences varying with time.

In addition, in other embodiments of the invention, the fatigue detection apparatus 100 further includes a storage unit and a warning unit. The storage unit is, for example, a hard disk or other type of storage medium, coupled to the processor 150 and configured to record a plurality of historical information. The historical information includes, for example, a plurality of users' various past physiological information, and so on, which reflects different physiological conditions of each of the users. However, the invention is not limited thereto. In this way, based on the aforesaid variation trend of the characteristic time differences varying with time, the processor 150 determines a fatigue detection result by corresponding the historical information to the physiological conditions of different users, thereby obtaining a more accurate determination result. On the other hand, the warning unit is coupled to the processor 150 and configured to issue a warning signal based on the determined fatigue detection result. The warning unit is, for example, a device capable of issuing an electrical stimulation signal, a sound signal, a visual signal or other warning signals. The invention is not limited thereto.

In an embodiment of the invention, the fatigue detection apparatus 100 is, for example, installed in a vehicle, so as to serve as a fatigue detection apparatus 100 for vehicles. FIG. 2 illustrates a schematic view of a fatigue detection apparatus for vehicles according to an embodiment of the invention. Referring to FIG. 2, the first detector 110 of the fatigue detection apparatus 100 for vehicles of the present embodiment is, for example, an ECG detector, including a first electrode 110_1 and a second electrode 110_2 configured to respectively detect potentials of left and right portions of the user's body, so as to obtain a complete ECG signal as a first physiological signal PS1. The second detector 130 of the fatigue detection apparatus 100 for vehicles is, for example, a PCG detector, installed on a seat belt SB so as to accurately obtain a PCG signal as a second physiological signal PS2 by using a characteristic of the seat belt SB of sticking to a human body. The processor 150 of the fatigue detection apparatus 100 for vehicles is installed, for example, on a seat belt buckle, on a vehicle console or in other positions in the vehicle. The invention is not limited thereto. In this way, by the fatigue detection apparatus 100 for vehicles provided by the embodiment of the invention, the fatigue detection result of the user during driving can be determined in real time. In other embodiments, the fatigue detection apparatus 100 for vehicles is further capable of, when determining in real time that the fatigue detection result of the user is a fatigue state, issuing a warning signal to alert the user in real time by the warning unit, so as to enhance driving safety.

However, the invention does not limit the installation position or use range of the fatigue detection apparatus 100. In other words, the fatigue detection apparatus 100 of the embodiment of FIG. 1 may also be installed in other positions or used in other situations according to user needs.

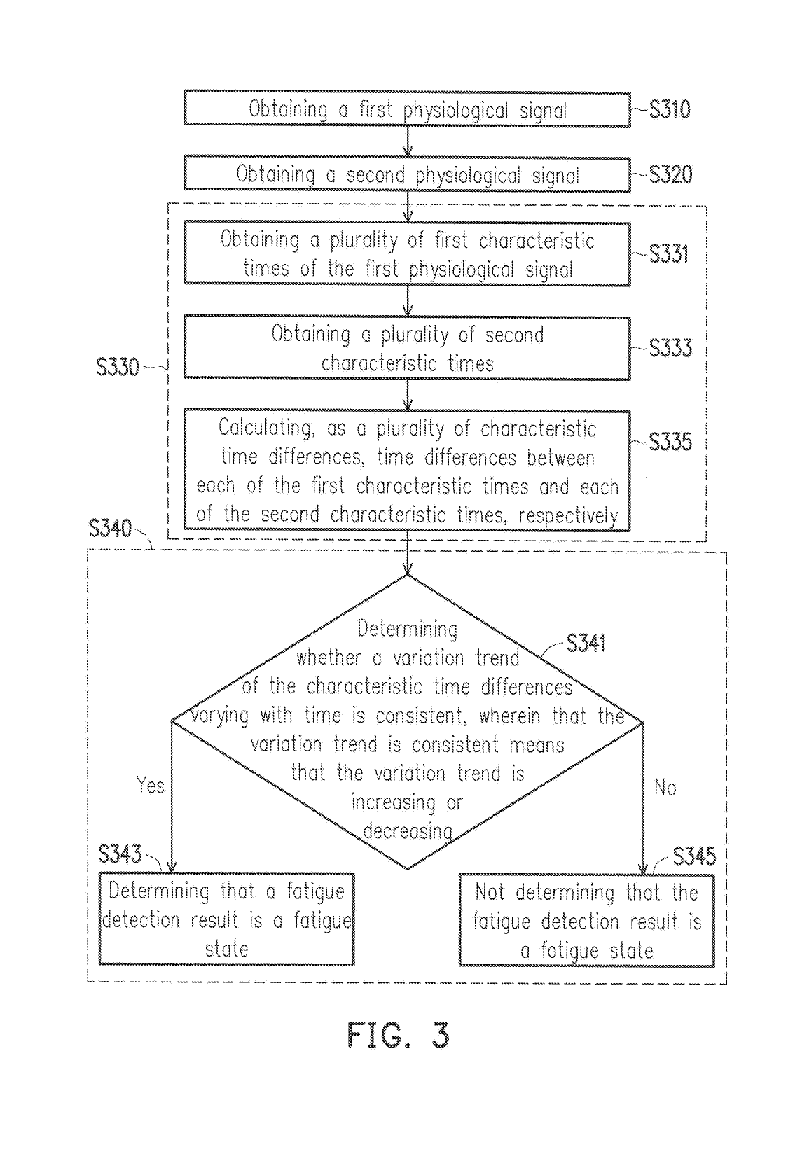

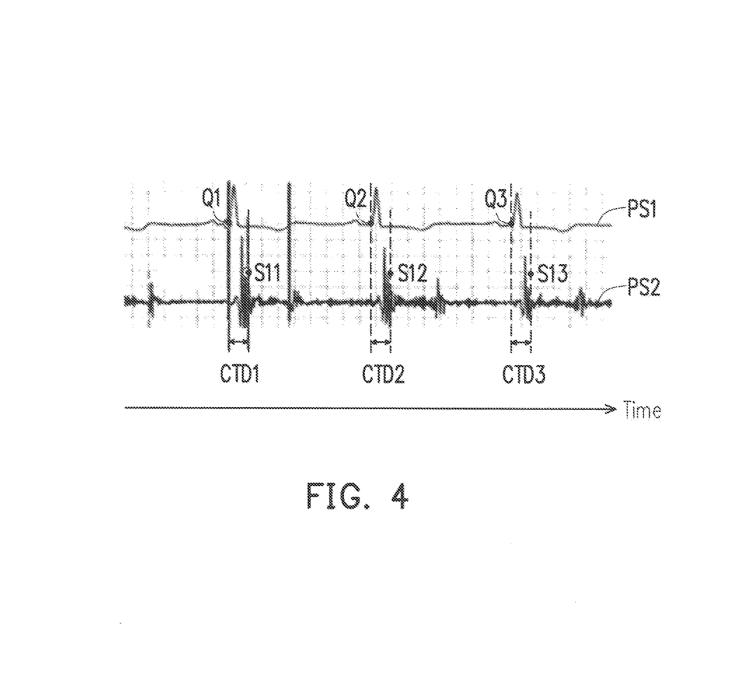

FIG. 3 illustrates a flow chart of a fatigue detection method according to an embodiment of the invention. FIG. 4 illustrates a schematic view of a fatigue detection method according to an embodiment of the invention. Referring to FIG. 1 to FIG. 4 together, the fatigue detection method of the present embodiment is applicable to the fatigue detection apparatus 100 of the embodiment of FIG. 1 or that of FIG. 2. Hereinafter, detailed steps of the fatigue detection method of the present embodiment are described with reference to the components of the fatigue detection apparatus 100 in FIG. 1.

First, the processor 150 of the fatigue detection apparatus 100 obtains the first physiological signal PS1 from the first detector 110 (step S310), and obtains the second physiological signal PS2 from the second detector 130 (step S320). In the present embodiment, the first physiological signal PS1 is, for example, an ECG signal, and the second physiological signal PS2 is, for example, a PCG signal, wherein how the first detector 110 and the second detector 130 respectively obtain the first physiological signal PS1 and the second physiological signal PS2 has been explained in detail in the embodiment of FIG. 1 and will not be repeated herein.

Then, the processor 150 obtains a plurality of characteristic time differences CTD1, CTD2 and CTD3 based on the first physiological signal PS1 and the second physiological signal PS2 (step S330). The characteristic time differences CTD1, CTD2 and CTD3 are, for example, pre-ejection periods (PEP) relevant to the first physiological signal PS1 and the second physiological signal PS2. In the present embodiment, the first physiological signal PS1 is an ECG signal having a plurality of QRS wave groups including a plurality of Q waves Q1 to Q3. The processor 150 obtains occurrence times of the Q waves Q1 to Q3 respectively as first characteristic times (step S331). On the other hand, the second physiological signal PS2 is a PCG signal having a plurality of first heart sounds S11 to S13. The processor 150 obtains occurrence times of the first heart sounds S11 to S13 respectively as second characteristic times (step S333). After obtaining the first characteristic times and the second characteristic times, the processor 150 calculates, as characteristic time differences, differences between each of the first characteristic times and each of the second characteristic times, respectively (step S335). In detail, in the present embodiment, the processor 150 calculates, as the characteristic time difference CTD1, a time difference between when the Q wave Q1 occurs and when the first heart sound S11 occurs, as the characteristic time difference CTD2, a time difference between when the Q wave Q2 occurs and when the first heart sound S12 occurs, and, as the characteristic time difference CTD3, a time difference between when the Q wave Q3 occurs and when the first heart sound S13 occurs.

After obtaining the characteristic time differences, the processor 150 determines a fatigue detection result based on a variation trend of the obtained characteristic time differences varying with time (step S340). In the present embodiment, the processor 150 determines whether the variation trend of the characteristic time differences CTD1, CTD2 and CTD3 varying with time is consistent (step S341), wherein that the variation trend is consistent means that the variation trend is monotonically increasing or decreasing. Generally, a length of time of the pre-ejection period reflects a cardiac output of the user. The longer the pre-ejection period, the less the cardiac output. As the cardiac output gradually decreases, so does cerebral blood flow, which causes fatigue to occur. Specifically, in an embodiment of the invention, the pre-ejection periods are used as the characteristic time differences CTD1, CTD2 and CTD3. When CTD3 is greater than CTD2, and CTD2 is greater than CTD1, it means that the variation trend of these characteristic time differences varying with time is increasing, which probably means that the cerebral blood flow of the user is gradually decreasing, and the processor 150 accordingly determines that the fatigue detection result is a fatigue state (step S343). Conversely, the processor 150 does not determine that the fatigue detection result is a fatigue state (step S345). In some embodiments, when determining that the fatigue detection result is the fatigue state, the processor 150 further issues a warning signal to alert the user by the warning unit. The method thereof has been explained in the embodiment of FIG. 1 and will not be repeated herein.

It should be noted that, to facilitate explanation, the numbers of the first characteristic time, the second characteristic time and the characteristic time difference in the present embodiment are three for exemplary purposes. However, the invention is not limited thereto. In other embodiments, according to needs, a larger or smaller number of characteristic times and characteristic time differences may be obtained for determining the fatigue detection result.

It is worth mentioning that, since the aforesaid embodiment uses the pre-ejection periods as the characteristic time differences, when the variation trend of the characteristic time differences varying with time is increasing, the processor 150 determines that the fatigue detection result is the fatigue state. However, in other embodiments, depending on different types of the obtained physiological signals, the type of the characteristic time difference obtained by calculation varies. Therefore, the invention does not impose any limitations on a correspondence relationship between the fatigue detection result and the variation trend of the characteristic time difference varying with time, and the correspondence relationship depends on, for example, the type of the obtained characteristic time difference. In other words, in other embodiments, the processor 150 may determine that the fatigue detection result is the fatigue state when a variation trend of another type of characteristic time difference varying with time is decreasing.

Particularly, in general, a decrease in heart rate shows only after the cerebral blood flow has decreased for a period of time. Therefore, the fatigue detection apparatus and the fatigue detection method provided by the embodiments of the invention use high level physiological indicators, and are capable of determining fatigue or issuing an alert in a more real-time manner than conventional techniques. On the other hand, since different users may have different physiological conditions, the embodiments of the invention determine whether the user is in the fatigue state based on whether the variation trend of the characteristic time difference varying with time is consistent, rather than based on an absolute numerical value of the characteristic time difference. In this way, the fatigue detection apparatus and the fatigue detection method provided by the embodiments of the invention are adapted for different users and determine fatigue detection results corresponding to the users, and thus have wider application.

In addition, in another embodiment of the invention, the processor 150 may, for example, determine a fatigue detection result by combining the variation trend of the characteristic time difference varying with time with a plurality of historical information. The historical information includes, for example, a plurality of users' various past physiological information, and so on, which reflects different physiological conditions of different users. That is, a determination criterion for determining a fatigue detection result may be decided based on a user's past physiological conditions, wherein the determination criterion may be, for example, the number of characteristic time differences having the same variation trend, or the like. The invention is not limited thereto. In this way, the invention is more adapted for different users and provides a more precise fatigue determination function.

In summary, the fatigue detection apparatus and the fatigue detection method provided by the embodiments of the invention determine a fatigue detection result of a user using the variation trend of the characteristic time difference varying with time, and are adapted for different users, so that accurate determination results are obtained from all of the different users. In addition, the characteristic time difference is relevant to the first physiological signal and the second physiological signal, and higher level physiological indicators may be obtained so as to enable more real-time fatigue determination, so that wider application may be achieved.

Although the invention has been disclosed with reference to the above embodiments, it will be apparent to persons of ordinary skill in the art that modifications to the described embodiments may be made without departing from the spirit of the invention. Accordingly, the scope of the invention will be defined by the attached claims and not by the above detailed descriptions.

What is claimed is:

1. A fatigue detection apparatus, comprising:
a first detector, configured to obtain a first physiological signal;
a second detector, configured to obtain a second physiological signal; and
a processor, coupled to the first detector and the second detector, wherein
the processor obtains a plurality of characteristic time differences based on the first physiological signal and the second physiological signal, and determines a fatigue detection result based on a variation trend of the characteristic time differences varying with time.

2. The fatigue detection apparatus according to claim 1, wherein the first physiological signal corresponds to a first physiological indicator, the second physiological signal corresponds to a second physiological indicator, and the first physiological indicator differs from the second physiological indicator.

3. The fatigue detection apparatus according to claim 1, wherein each of the characteristic time differences is relevant to the first physiological signal and the second physiological signal.

4. The fatigue detection apparatus according to claim 1, wherein if the variation trend of the characteristic time differences varying with time is consistent, the processor determines that the fatigue detection result is a fatigue state, wherein that the variation trend is consistent means that the variation trend is either increasing or decreasing.

5. The fatigue detection apparatus according to claim 1, further comprising:
a storage unit, coupled to the processor and configured to record a plurality of historical information, wherein
the processor determines the fatigue detection result based on the historical information and the variation trend.

6. The fatigue detection apparatus according to claim 1, further comprising:
a warning unit, coupled to the processor and configured to issue a warning signal based on the fatigue detection result.

7. The fatigue detection apparatus according to claim 2, wherein the first detector is an electrocardiogram (ECG) detector, and the second detector is a phonocardiogram (PCG) detector.

8. The fatigue detection apparatus according to claim 7, wherein the first physiological signal is an ECG signal, the second physiological signal is a PCG signal, and the characteristic time differences are a plurality of pre-ejection periods (PEP).

9. The fatigue detection apparatus according to claim 1, adapted to be installed in a vehicle so as to serve as a fatigue detection apparatus for vehicles, wherein at least one of the first detector and the second detector is installed on a seat belt of the vehicle.

10. A fatigue detection method, comprising:
obtaining a first physiological signal;
obtaining a second physiological signal;
obtaining a plurality of characteristic time differences based on the first physiological signal and the second physiological signal; and
determining a fatigue detection result based on a variation trend of the characteristic time differences varying with time.

11. The fatigue detection method according to claim 10, wherein the first physiological signal corresponds to a first physiological indicator, the second physiological signal corresponds to a second physiological indicator, and the first physiological indicator differs from the second physiological indicator.

12. The fatigue detection method according to claim 10, wherein step of obtaining the characteristic time differences based on the first physiological signal and the second physiological signal comprises:
obtaining a plurality of first characteristic times of the first physiological signal;
obtaining a plurality of second characteristic times of the second physiological signal; and
calculating, as the characteristic time differences, time differences between each of the first characteristic times and each of the second characteristic times, respectively.

13. The fatigue detection method according to claim 10, wherein step of determining the fatigue detection result based on the variation trend of the plurality of characteristic time differences varying with time comprises:
determining whether the variation trend of the characteristic time differences varying with time is consistent; and
if the variation trend of the characteristic time differences varying with time is consistent, determining that the fatigue detection result is a fatigue state, wherein
that the variation trend is consistent means that the variation trend is either increasing or decreasing.

14. The fatigue detection method according to claim 10, wherein step of determining the fatigue detection result based on the variation trend of the characteristic time differences varying with time comprises:
determining the fatigue detection result based on a plurality of historical information and the variation trend of the characteristic time differences varying with time.

15. The fatigue detection method according to claim 10, further comprising:
issuing a warning signal based on the fatigue detection result.

16. The fatigue detection method according to claim 11, wherein the first detector is an electrocardiogram (ECG) detector, and the second detector is a phonocardiogram (PCG) detector.

17. The fatigue detection method according to claim 16, wherein the first physiological signal is an ECG signal, the second physiological signal is a PCG signal, and the characteristic time differences are a plurality of pre-ejection periods (PEP).
